(19) United States
(12) Patent Application Publication
(10) Pub. No.: US 2008/0161741 A1
(43) Pub. Date: Jul. 3, 2008

(54) OCULAR IMPLANT AND METHODS FOR MAKING AND USING SAME

(75) Inventors: Eric A. Bene, Lynn, MA (US); Tim J. Morrill, Plaislow, NH (US); Margaret B. Mulhern, Groton, MA (US); Thaddeus L. Wandel, Croton, NY (US); Jon B. Taylor, Groton, MA (US); Leon Mir, Andover, VT (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: Becton, Dickinson and Company, Franklin Lakes, NJ (US)

(21) Appl. No.: 12/050,346

(22) Filed: Mar. 18, 2008

Related U.S. Application Data
(63) Continuation of application No. 10/857,452, filed on Jun. 1, 2004.
Application No. 10/857,452 is a continuation-in-part of application No. 10/182,833, filed on Dec. 27, 2002.
(60) Provisional application No. 60/175,658, filed on Jan. 12, 2000.

(51) Int. Cl. A61F 9/00; A61M 27/00

(52) U.S. Cl. 604/9

(57) ABSTRACT

An ocular implant device that is insertable into either the anterior or posterior chamber of the eye to drain aqueous humor and/or to introduce medications. The implant can include a substantially cylindrical body with a channel member that regulates the flow rate of aqueous humor from the anterior chamber or introduces medications into the posterior chamber, and simultaneously minimizes the ingress of microorganisms into the eye.

### CROSS-REFERENCE TO RELATED APPLICATIONS

This application is a continuation of and claims priority under 35 U.S.C. §120 to U.S. application Ser. No. 10/857,452, filed Jun. 1, 2004, which is a continuation-in-part of, and claims priority under 35 U.S.C. §120 to, U.S. patent application Ser. No. 10/182,833, filed Dec. 27, 2002, which is the national stage of International Application No. PCT/US01/00350, filed Jan. 5, 2001, which claims the benefit of U.S. provisional patent application Ser. No. 60/175,658, filed Jan. 12, 2000, the entire content of each being incorporated herein by reference. International Application No. PCT/US01/00350 was published under PCT Article 21(2) in English.

### FIELD OF THE INVENTION

The present invention relates to an ocular implant and more particularly, a filtered and/or flow restricting ocular implant for use through the cornea of an eye to relieve intraocular pressure, and for use through the sclera to introduce medications into the posterior chamber of the eye. In doing so, the embodiments of the present invention are applicable for both transcorneal and transscleral applications.

### BACKGROUND OF THE INVENTION

Glaucoma, a condition caused by optic nerve cell degeneration, is the second leading cause of preventable blindness in the world today. A major symptom of glaucoma is a high intraocular pressure, or “IOP”, which is caused by the trabecular meshwork failing to drain enough aqueous humor fluid from within the eye. Conventional glaucoma therapy, therefore, has been directed at protecting the optic nerve and preserving visual function by attempting to lower IOP using various methods, such as through the use of drugs or surgery methods, including trabeculectomy and the use of implants.

Trabeculectomy is a very invasive surgical procedure in which no device or implant is used. Typically, a surgical procedure is performed to puncture or reshape the trabecular meshwork by surgically creating a channel thereby opening the sinus venosus. Another surgical technique typically used involves the use of implants, such as stems or shunts, positioned within the eye and which are typically quite large. Such devices are implanted during any number of surgically invasive procedures and serve to relieve internal eye pressure by permitting aqueous humor fluid to flow from the anterior chamber, through the sclera, and into a conjunctive bleb over the sclera. These procedures are very labor intensive for the surgeons and are often subject to failure due to scaring and cyst formations.

Another problem often related to the treatments described above includes drug delivery. Currently there is no efficient and effective way to deliver drugs to the eye. Most drugs for the eye are applied in the form of eye drops which have to penetrate through the cornea and into the eye. Drops are a very inefficient way of delivering drugs and much of the drug never reaches the inside of the eye. Another treatment procedure includes injections. Drugs may be injected into the eye, however, this is often traumatic and the eye typically needs to be injected on a regular basis.

One solution to the problems encountered with drops and injections involves the use of a transcornea shunt. The transcornea shunt has also been developed as an effective means to reduce the intraocular pressure in the eye by shunting aqueous humor fluid from the anterior chamber of the eye. The transcornea shunt is the first such device provided to drain aqueous humor fluid through the cornea, which makes surgical implantation of the device less invasive and quicker than other surgical options. Additional details of shunt applications are described in International Patent Application No. PCT/US01/00350, entitled “Systems And Methods For Reducing Intraocular Pressure”, filed on Jan. 5, 2001 and published on Jul. 19, 2001 under the International Publication No. WO 01/50943, the entire content of which is incorporated herein by reference.

As noted in the Application No. PCT/US01/00350 above, however, existing shunts are also subject to numerous difficulties. The first problem associated with shunt use is the regulation of aqueous outflow. This problem typically results because the drainage rate of the fluid depends substantially on the mechanical characteristics of the implant until there has been sufficient wound healing to restrict fluid outflow biologically. Effective balancing of biological and mechanical resistance to aqueous humor outflow remains a problem for implant-based drainage procedures. Prior devices utilize a variety of mechanisms to restrict such aqueous outflow. Each of these mechanisms, however, may become a liability once wound healing has been established. Restrictive elements within the implant, when combined with the restriction effected by wound healing, may inordinately reduce the rate of aqueous humor outflow possibly to non-therapeutic levels.

The second problem associated with existing shunt use is the possibility of intraocular infection. Unfortunately, the presence of an implant provides a conduit through which bacteria can gain entry to the anterior chamber, thereby resulting in intraocular infections. Certain drainage devices have introduced filters, valves or other conduit systems which serve to impede the transmission of infection into the anterior chamber, however, these mechanisms have limitations. Even when effective in resisting the transit of microorganisms, they have hydraulic effects on fluid outflow that may also impair effective drainage.

Finally, a problem of local tissue tolerance arises with existing devices because the implant, as a foreign body, may incite tissue reactions culminating in local inflammation or extrusion. This may be perceptible or uncomfortable for the patient, and these reactions to the presence of the implant may make its use clinically unsuitable.

Accordingly, a need exists for a transcornea shunt or implant for use in providing controlled anterior chamber drainage while limiting ingress of microorganisms. Still further, a need exists for a device and method to allow drugs to be transmitted to the eye through the cornea over a prolonged period of time such that repeated injury to the eye does not occur as commonly associated with repeated injections, and still further allows a slow continuous infusion into the eye.

### SUMMARY OF THE INVENTION

It is therefore an object of the present invention to provide a device and method that may be used to relieve IOP by draining the anterior chamber of the eye of aqueous humor fluid in a controlled manner.

It is another object of the present invention to provide a device and method that may be used to communicate a substance, such as a medication, into the posterior chamber of the eye.

It is yet another object of the present invention to provide a device and method that may be used as an implant having a size, shape and composition suitable for various applications, and including one or more filters, valves or restrictors to configure a desired response provided by the implant.

These and other objects are substantially achieved by providing an implant that is insertable through the clear cornea of the eye into the anterior chamber to drain aqueous humor, or similarly insertable through the sclera to introduce medications into the posterior chamber of the eye. The implant may include a substantially cylindrical body having one or more channels that permits drainage of aqueous humor from the anterior chamber to the external surface of the clear cornea, or permits substance release into the posterior chamber of the eye. The implant may further include a head that rests against an outer surface of the clear cornea or sclera, a foot that rests against an inner surface of the cornea or sclera, and one or more elongated filter members retainable within the channel of the body to regulate the flow rate of aqueous humor, introduce medications, and minimize the ingress of microorganisms.

### BRIEF DESCRIPTION OF THE DRAWINGS

The above and other objects and advantages will be apparent upon consideration of the following drawings and detailed description. The preferred embodiments of the present invention are illustrated in the appended drawings in which like reference numerals refer to like elements and in which:

FIG. 1 is an enlarged perspective view of an example implant in accordance with an embodiment of the present invention;

FIG. 2 is an enlarged cross-sectional view of an example implant in accordance with an embodiment of the present invention;

FIG. 3 is another enlarged cross-sectional view of the implant of FIG. 2;

FIGS. 4-15 are enlarged cross-sectional views of several example implants in accordance with an embodiment of the present invention;

FIGS. 16-19 are enlarged cross-sectional views of several installed example implants in accordance with an embodiment of the present invention;

FIGS. 20-22 are enlarged cross-sectional views of several example implants in accordance with an embodiment of the present invention;

FIGS. 23-24 are enlarged cross-sectional views of several installed example implants in accordance with an embodiment of the present invention;

FIGS. 25-28 are enlarged perspective views of an example implant in accordance with an embodiment of the present invention;

FIGS. 29-36 are enlarged cross-sectional views of several example implants in accordance with an embodiment of the present invention;

FIGS. 37A-37B are enlarged cross-sectional views of an example capillary filter in accordance with an embodiment of the present invention;

FIGS. 37C-37D are enlarged cross-sectional views of an example hollow fiber element as provided in the filter of FIG. 37A;

FIGS. 38-42 are enlarged cross-sectional views of several additional example capillary filters in accordance with an embodiment of the present invention; and

FIGS. 43-45 are enlarged cross-sectional views of an exemplary implant which can include any features of FIGS. 1 through 42 in accordance with an embodiment of the present invention.

In the drawing figures, it will be understood that like numerals refer to like structures.

### DETAILED DESCRIPTION OF THE PREFERRED EMBODIMENTS

The transcornea shunt or implant (hereinafter “shunt”) has been developed to serve several purposes, such as to reduce the intraocular pressure (IOP) in the eye by shunting aqueous humor fluid from the anterior chamber of the eye, through the cornea, and to the terafilum. To do so, the shunt must be implanted through a small incision and into the cornea of the eye, actually extending between the inner and outer surface of the cornea. In yet another application, the shunt can be implanted through the sclera to introduce a substance into the posterior chamber of the eye.

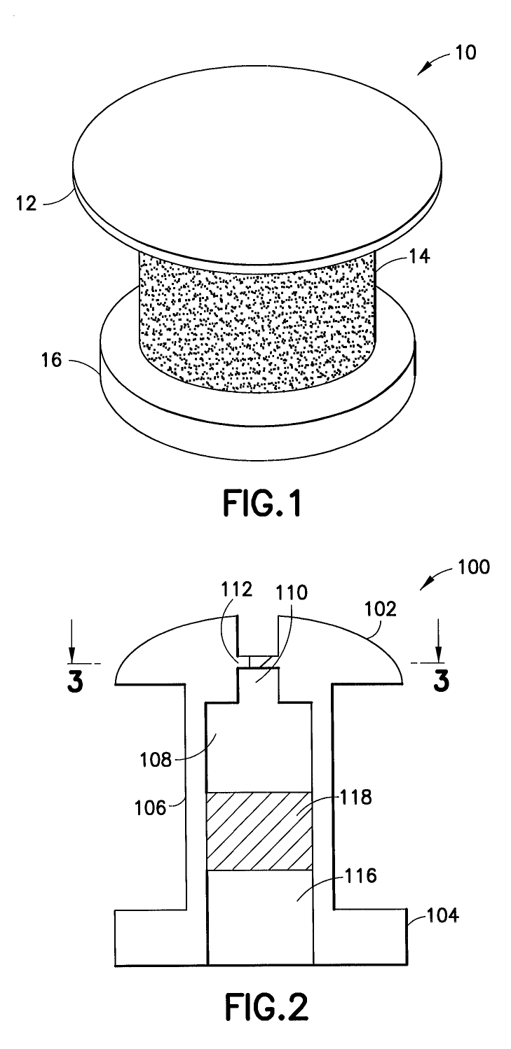

As shown in FIG. 1, an enlarged perspective view of a shunt according to an embodiment of the present invention may be seen. In a representative embodiment, the shunt may be approximately one millimeter long with an outer diameter of approximately 0.5 mm. While the shunt illustrated in this figure is shown as a cylindrical structure, it is understood that other shapes of tubular conduits may be suitable as well. For example, the shunt may assume an oval or irregular shape as described in greater detail below.

FIG. 1 shows the shunt 10 dimensionally adapted for transcorneal positioning. The head 12 is located on the external or epithelial surface of the cornea when the shunt is in position within the cornea. As shown in this figure, the head 12 may be dome-shaped to provide a continuous transition surface from the device to the cornea. This shape may also be well tolerated by the patient's eyelid. While this shape appears particularly advantageous, other shapes of the head may be designed to provide the same advantages. For example, a minimally protruding flat head with rounded edges may be equally well tolerated. The undersurface (not shown) of the head 12 may be flat or curved suitably to match the shape of the corneal surface whereupon the device is to be positioned. The head 12, the body 14 and the foot 16 may all be formed integrally as a unit, or the head or the foot may be formed integrally with the body.

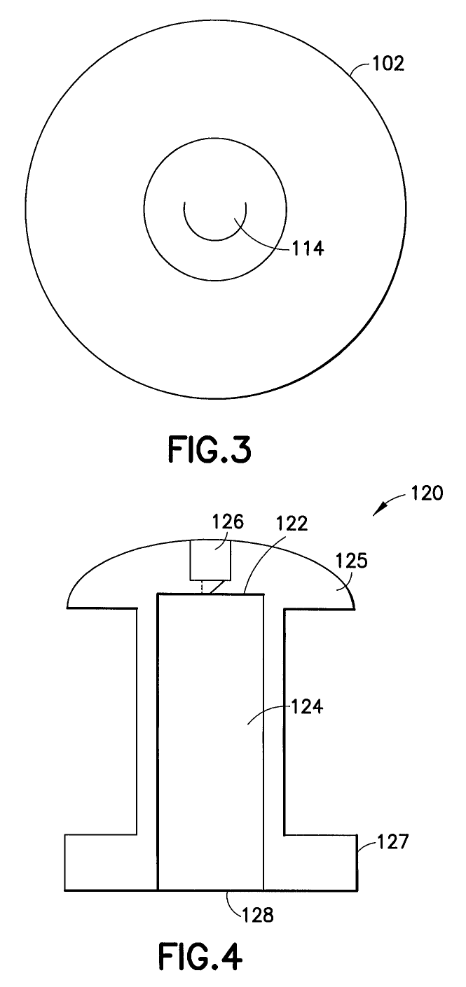

In a first embodiment of the present invention as shown in FIGS. 2 and 3, a shunt 100 is shown having a distal and proximal end comprising a head 102 and foot 104, respectively, between which extends a body 106. An opening 108 is provided between the distal and proximal ends for allowing fluid communication. The opening includes a narrowed portion 110 in which a thin layered flap extends as shown more clearly in the cross sectional view in FIG. 3. A solid member 112 covers the narrowed portion 110, and includes the flap 114 having a substantially semi-circular shape which maintains the flap in a closed position until a minimal pressure is applied from the distal direction of the opening. The flap then opens and allows regulated flow from the distal to the proximal end of the opening.

As used herein, the term “proximal” refers to a location on any device farthest from the patient in connection with which the device is used. Conversely, the term “distal” refers to a location on the device closest to the patient in connection with which the device is used.

The flap 114 is constructed of a material such as hydrogel, to allow the flap to easily open. The flap circumference is contoured to allow the flap to open in one direction only, thereby preventing a reverse flow from the proximal to the distal end of the opening. Specifically, the flap 114 can be constructed having a tapered, or sloped outer circumference which is used to mate with a similar surface about an inner circumference of the opening 108. The tapered surfaces, shown more clearly in the cross-sectional view of FIG. 2, restricts the flap opening to a single direction and serves to prevent the ingress of microorganisms into the opening 108.

The opening also includes a wider portion 116 in which a filter 118 can be positioned. The filter can comprise any number of filters as known to those skilled in the art, or include an improved filter mechanism as described in greater detail below.

In the embodiment shown in FIG. 2, the flap 114 and filter 118 together form a fluid shunt between the exterior and interior of the eye surface. The filter and shunt body can be constructed in a number of fashions in accordance with various embodiments of the present invention. For example, the filter 118 can be constructed as the shunt (i.e. the filter body is substantially solid and serves as the actual shunt). In yet another embodiment, an opening provided in the head of the shunt can serve as the filter (i.e. task specific valve mechanism).

As shown in the shunt 120 of FIG. 4, an opening, or one-way valve 122, is provided between the narrow and wide portion, 126 and 128, respectively, of the opening 124. In the embodiment shown in FIG. 4, no filter is provided and the valve 122 controls flow from the distal to proximal end, and prevents a reverse flow within the opening. As with the flap 114 shown in FIGS. 2 and 3, the one-way valve 122 can be constructed having a tapered, or sloped surface which is used to mate with a similar surface about an inner circumference of the opening 124. The tapered surfaces restrict the one-way valve opening to a single direction and serves to prevent the ingress of microorganisms into the opening 124.

In the embodiments of the present invention described below, the filters, such as the filter 118 of FIG. 2, can be comprised of ceramic, coral, stainless steel, titanium, silicone or PHEMA (i.e. poly 2-hydroxyethylmethacrylate), and any number of polymer materials, depending upon the specific tasks required. In addition to stainless steel, any metal which can provide more consistent filters may be used. Metals, or similar materials which are bacteria resistant to some degree, such as silver or platinum can also be used. The device, filter, or combination, can incorporate a number of such antimicrobial agents as a coating, impregnated material, or construction material, including ionic metal compounds, such as copper, zinc or silver (i.e., vapor deposition silver plating); antibacterial polymers (i.e., nonsoluble deposited via a loss salt method), such as PHMB (polyhexamethyl biguanide) and liquid crystal polymers; organic compounds, such as alkyl trypsin, biguanide, triclosan, and CHG (chlorhexidine); infused bacteria intolerant substances and inorganic compounds, such as quaternary ammonium salt and metal oxides.

The filter can also be constructed of titanium, which can be further oxidized to increase hydrophilicity and improve flow rates, as air bubbles will be less likely block the filter. Still other filter materials can include soluble/insoluble glass containing an antimicrobial, in which the glass dissolves and is replaceable. An example of an insoluble glass material would be glass frit made up of glass fibers or granules.

Such filters may also be constructed of glass spheres which are vacuum plated with an antimicrobial substance. Such spheres can be allowed to move within larger openings, or provided as a filter constructed of bonded spheres, and can further include a silver ion that is time release impregnated in such glass soluble spheres. A number of 3.5 micron spheres will produce a 0.5 micron hole when secured with a substance, such as a cellulose binder.

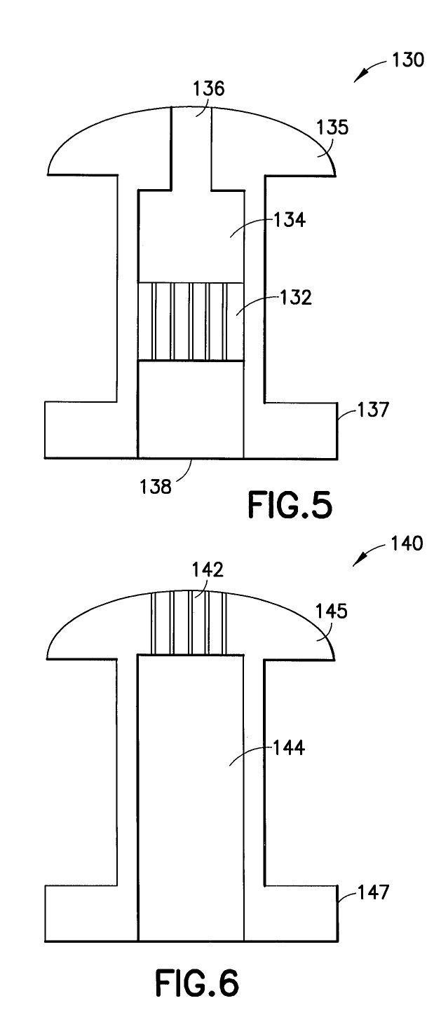

The filter can also be constructed as a flow restrictor, such as a glass capillary flow restrictor 132 as shown in FIG. 5 which includes multiple through holes that are used to effectively control flow between the distal and proximal ends of the opening 134 in the shunt 130. In addition to controlling flow, the multiple through holes can be used to prevent bacterial infiltration. As shown in shunt 140 of FIG. 6, such a capillary flow restrictor configuration 142 can also be incorporated into the head, or cap 145, located at the proximal end of the opening. In such an embodiment, the cap portion covering the opening can be provided with a multiple through hole section 142 to control the flow and prevent bacteria infiltration, and the filter (i.e. 118 and 132), can be eliminated. Each through hole of section 142, whether provided as a plurality, or as a single through hole, can be surrounded by an antimicrobial in a surrounding tube, and still further provided with very smooth surfaces.

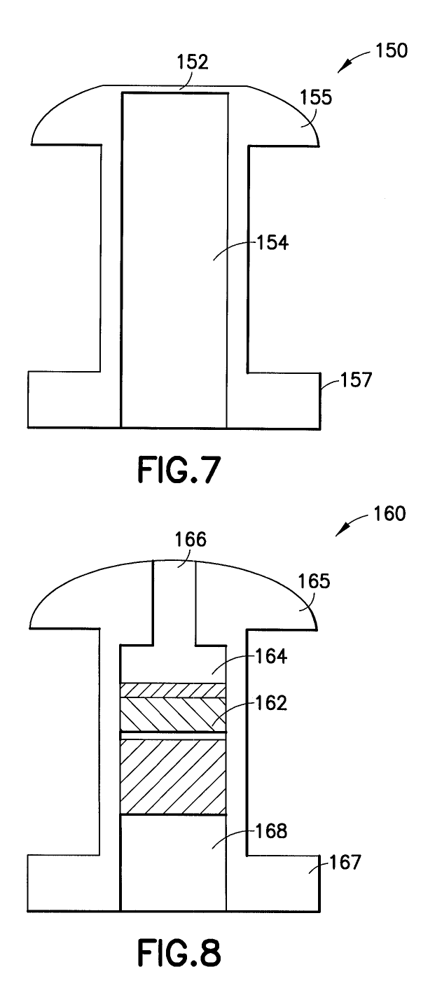

In still another embodiment of the present invention shown in FIG. 7, the cap 155 portion covering the opening 154 of shunt 150 can be constructed of a membrane 152, such as a porous hydrogel membrane to control flow (i.e. controlled diffusion) and prevent bacteria infiltration, and the filter (i.e. 118 and 132), can be eliminated. The hydrogel can also be provided to allow epithelium to grow over the cap 155 portion, resulting in the membrane 152. An epithelium membrane can allow fluid to diffuse and prevent bacterial infiltration.

In each embodiment described above in which a filter, membrane or capillary cap portion is used, multiple components can be used in cooperation. As shown in the shunt 160 of FIG. 8, stacked filters 162, including two or more separate filters or screens of varying pore sizes and construction, and varying cap construction configurations, can be used in cooperation. The selection and combination of stacked filters can be used to optimize flow control and bacterial infiltration. For example, the stacked filters 162 can be comprised of one or more drilled and stacked plates, glass disks in a tube, silicon stacks, or silver plates, fibers or screens, wherein each may be provided with through holes of various diameters, or slotted openings providing increased flow rates. Spacing and positioning of the stacks can be used to create biotraps, multiple chambers, tortuous paths (i.e. coil paths), tubes or channels. Still further, the plates can consist of grooved or etched plates, or etched layers of plates having still further unique structures, such as a honeycomb configuration. Likewise, the plates can be constructed of materials which can be arranged to create a semiconductor grid or polarizer.

The shunt body itself can be constructed of any number of materials, including but not restricted to ocular hydrogel (i.e., poly hydroxyethyl methacrylate-methacrylic acid copolymer (polyHEMA-MAA), polyHEMA, copolymers and other expansion material hydrogels), silicone, PMMA (i.e. polymethylmethacrylate), hylauronic acid, silicone/hydrogel combinations, silicone acrylic combinations and fluorosilicone acrylates. Such silicone materials have higher strength and include a larger degree of beneficial oxygen permeability and exhibit a high degree of protein and lipid deposition resistance. The use of silicone combinations, such as silicone/hydrogel combinations, further combines the advantages of each.

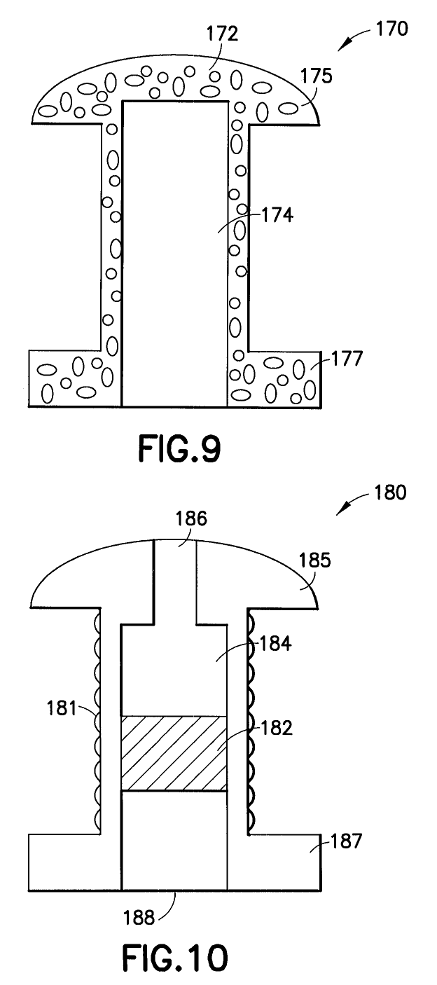

The construction materials of the shunt body can be selected from materials above and fabricated in any number of fashions in accordance with the embodiments of the present invention. For example, a shunt body 170 can be constructed in a porous manner as shown in FIG. 9, in which a filter is not required. The porous material of the shunt body itself serves as a filter and/or fluid communication means, and the selection of materials, based upon available pore sizes, can be used to effectively construct a shunt body that functions as an effective filter for a specific application. Still other shunt construction materials can be selected to include coatings of agents applied externally to the shunt. These agents, such as silver nitrate, can be used to minimize neovascularization and protein deposition, or serve as an antibacterial. The shunt body can also be provided with a coating agent and/or a surgical adhesive, such as Bioglue®, available from Cryolife Inc. located in Kennesaw, Ga., fibrin-based glue, marine adhesive proteins (i.e. algae), and synthetic polymeric adhesive such as cyanoacrylate.

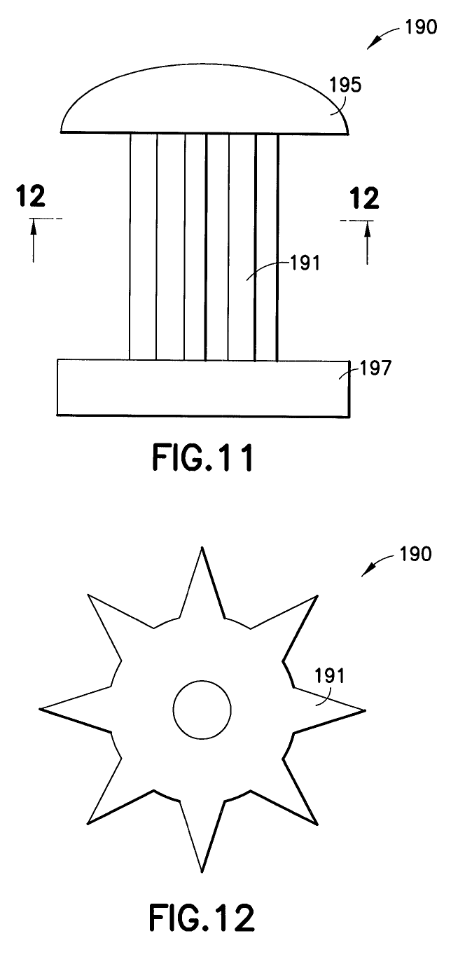

Any of the above described materials can be used in various combinations to create a shunt body having two or more levels of surface roughness or texture. For example, as shown in FIG. 10, the proximal end 185 of the shunt 180 can be constructed to include a smooth surface for comfort on the cornea and eyelid, while the shunt body 181 extending between the distal and proximal ends can be constructed having a rough surface for strong cellular attachment. In addition to having two or more levels of surface roughness, each embodiment can also include a shunt body extending between the distal and proximal ends that is substantially round, oval or irregular shaped, such as star shaped as shown in FIGS. 11 and 12. An irregular cross-section, such as the star-shaped cross section of shunt 190, allows better securement of the shunt in the eye. The use of a variable shaped shunt body cross section further allows the use of a number of incision patterns, such as an X-shaped, O-shaped, and T-shaped incision. Once construction materials are selected, a number of shunt body shapes can be used to effectively implement the embodiments of the present invention.

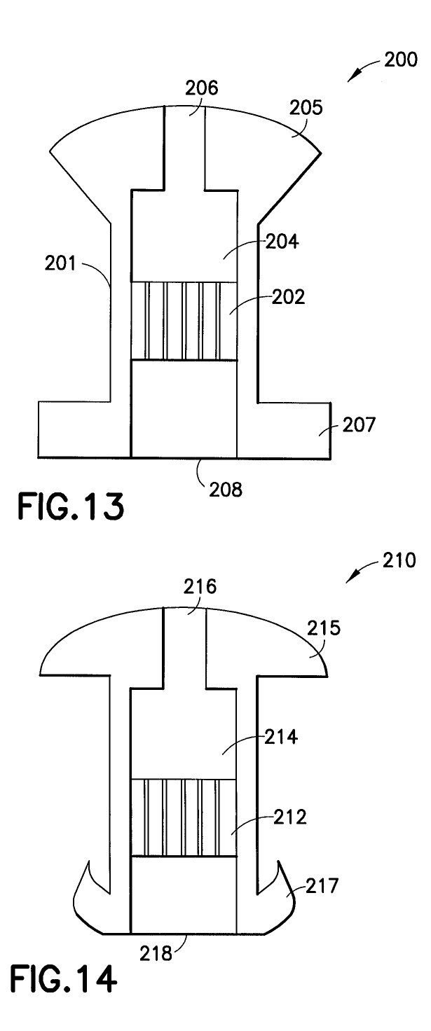

As noted above, the shunt body extending between the distal and proximal ends can be substantially round, oval or irregular shaped. As shown in FIG. 13, the shunt 200 can also be constructed having irregular shaped distal and/or proximal ends 207 and 205, respectively, to serve specific applications. For example, as shown in FIG. 13, the shunt cap 205 is constructed having a martini glass shape. This, and similar shapes can be effectively used to prevent shunt extrusion and are generally more comfortable on the eye as each minimizes foreign body sensation. Additionally, such a shape exhibits less leakage after initial implantation. In so constructing the device, the cap, or proximal end of the shunt can be overmolded to provide a smoother finish.

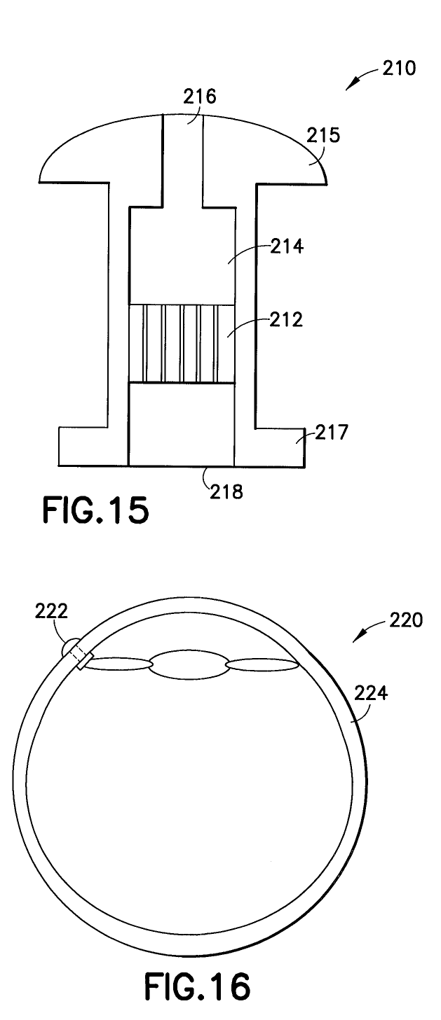

Yet another shape in accordance with an embodiment of the present invention is shown in FIGS. 14 and 15. The shunt 210 includes a distal and proximal end in which the distal end 217 deforms during, and subsequently after implantation. In this case, installation requires a smaller incision, as the inserted distal end 217 is deformable, or reduced to a smaller shape during installation as shown in FIG. 14. As shown in FIG. 15, after successfully reaching the inner surface, the distal end 217 expands to a larger size upon hydration or exposure to body temperature. Such a configuration allows easier implantation.

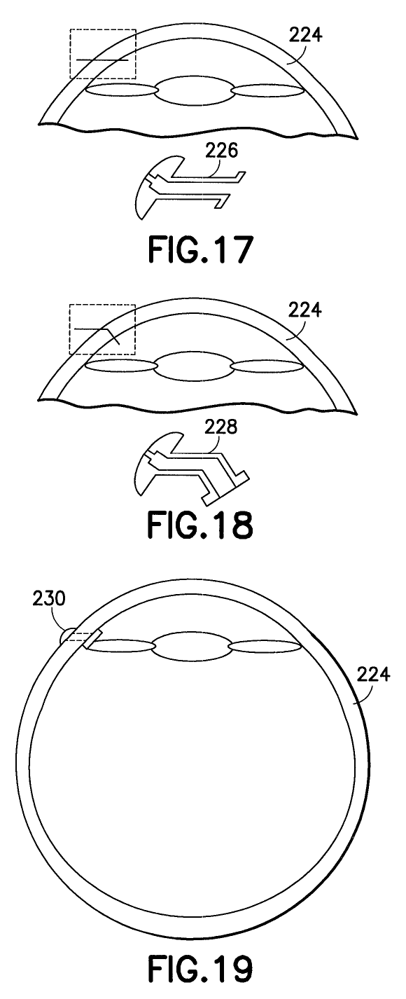

The shape can also be conformed to an insertion position as shown in FIG. 16. As known to those skilled in the art, shunt implantation can occur at the sclera cornea junction. At such implantation sites, the distal and proximal ends of the shunt 222 can be beneficially constructed at an angle relative to the shunt body extending therebetween. The relative angle of the embodiment shown in FIG. 16 can be further modified as shown in shunts 226 and 228 of FIGS. 17 and 18, respectively, for specific site locations, such as clear cornea insertions. Consideration can be given in such installation applications to an ability to lock the shunt in place. Specifically, the placement of the shunt at the limbus (e.g. the margin of the cornea overlapped by the sclera) can function to lock the distal end, or foot of the shunt in place as shown in FIG. 19.

As noted above, the shunt body can also be provided with a coating agent, such as a surgical adhesive. The use of a surgical adhesive during the implantation procedure can ensure sealing and/or secure the placement of the shunt. A still more effective use of a surgical adhesive is provided where a stitch is used with the implantation procedure. For example, currently the implantation procedure requires the creation of an approximately 1.5 to 1.6 mm incision into which the distal end, or foot of the shunt is placed. In an alternate method, the procedure can require an incision and a suture to secure the shunt into place.

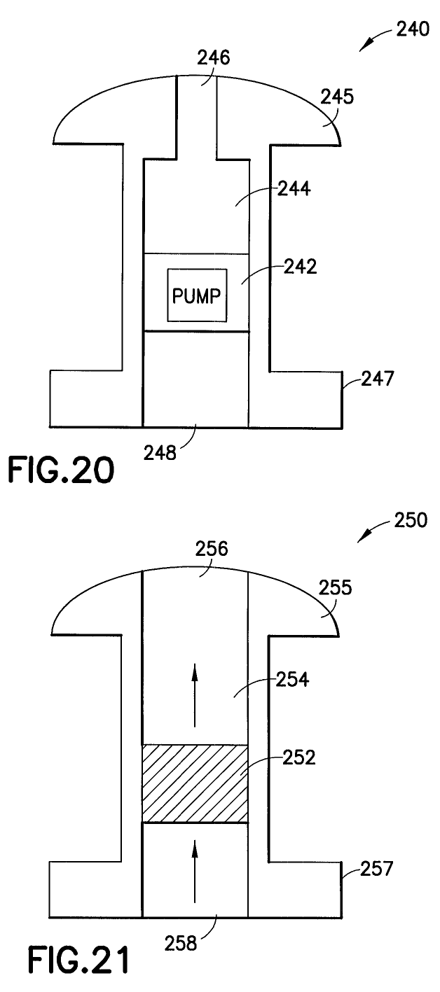

The filters provided in the embodiments described above can also be provided in addition with any number of micro-devices, such as a micro-mechanical pump 242 as shown in the shunt 240 of FIG. 20. Such technologies and devices can also be used to replace the filters, valves and restrictors described above.

The filter, restrictor and/or micro-device in each embodiment described above can be permanent, removable and/or replaceable. Therefore, the user has the option of using a shunt having a removable and replaceable filter, such that if the filter clogs the filter can be changed, thereby preventing the required replacement of the entire shunt. For example, as shown in FIG. 21, the filter 252 of shunt 250 can be simply pushed from the opening and replaced. Such a replacement can occur when a filter is clogged, or at any regular interval to maintain a performance level. Replacement can also occur when the user desires to change the flow rate or flow characteristics of the shunt. A replacement can also occur when a filter is used to introduce a medication into the eye.

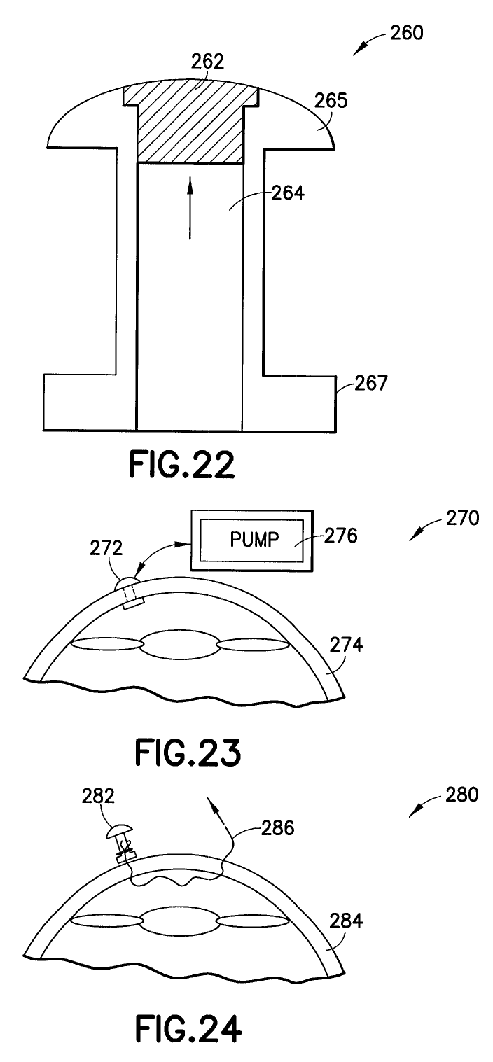

The replaceable filter described above can be constructed in a fashion to ease replacement, installation and identification in a number of ways. As shown in FIG. 22, the opening at the head 265 of the shunt 260 can be constructed having a countersunk entry at opening 264, which prevents the filter from traveling an uncontrolled distance into the opening and provides for easier removal and replacement from the proximal end of the shunt.

In yet another embodiment of the present invention which provides for easier insertion, a shunt includes a coupling mechanism for use with a device, such as an external pump. In the embodiment shown in FIG. 23, the shunt 272 is constructed to be expandable. Once positioned in a small incision in the eye 274, an external pump 276 can be used to expand the shunt 272 after implantation. The shunt therefore, can be smaller prior to expansion, thereby requiring a smaller incision for easier implantation. Also, the expanded shunt 272 more effectively fills leak gaps. As shown in FIG. 24, the shunt 282 as described above can be implanted using a suture 286 to pull the shunt through an incision and into the cornea 284. Still other implantation techniques include shooting the shunt into a proper implantation position. The construction of the shunt can be adapted to allow implantation using such techniques, in addition to removal techniques using any number of devices, such as a phacoemulsification machine.

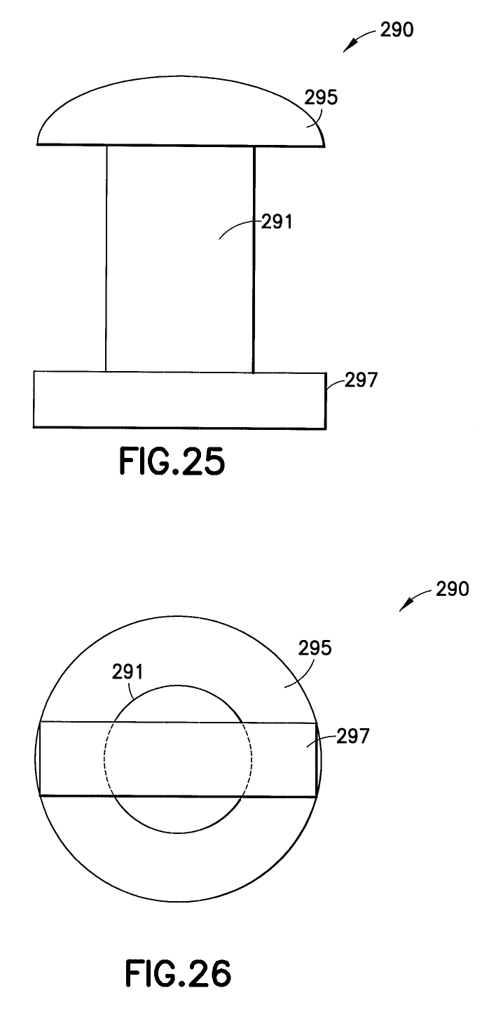

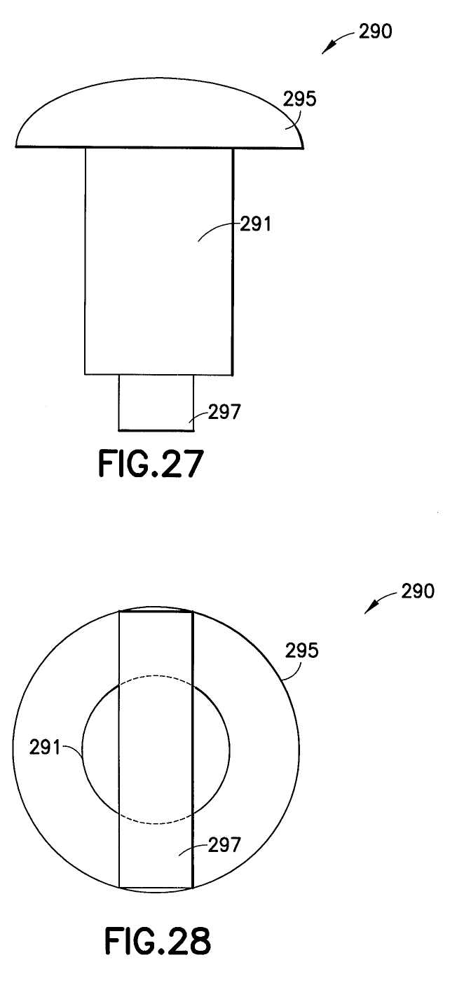

In yet another embodiment, the shunt 290 can be constructed having a linear distal portion 297 as shown in FIGS. 25 through 28. The linear distal member 297 replaces the round distal member of the embodiments described above. This allows greater ease in insertion into a typically linear incision. Upon insertion, the shunt 290 can be turned substantially 90 degrees to displace the linear distal member 297 perpendicular to the incision axis thereby securing the shunt 290.

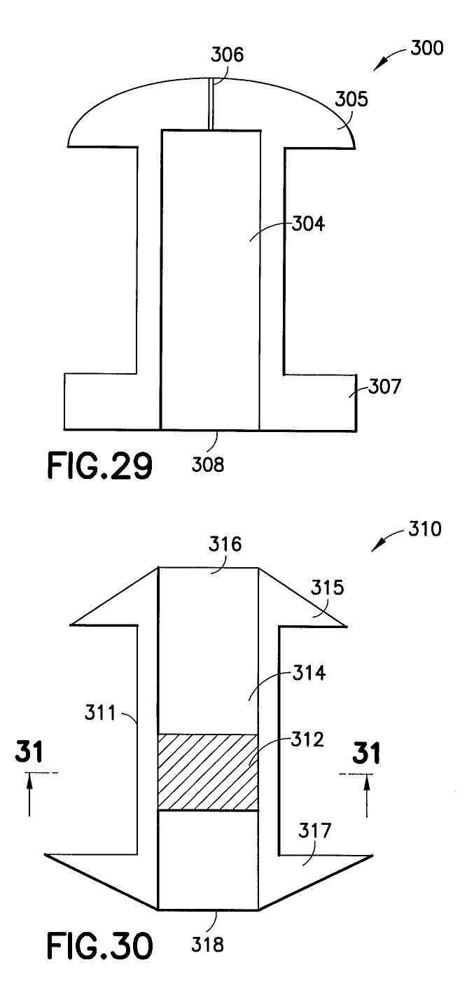

The various embodiments described above can be used to construct a shunt adaptable to any number of purposes, such as procedures allowing IOP reduction after cornea transplant procedures or cataract surgery. It can also be used for veterinary and cosmetic uses, and relieving dry eye conditions. The shunt body can also be used essentially as a catheter for the eye. As shown in FIG. 29, the proximal end 305 of the shunt opening 304 can be covered, sealed or provided as a slit to create a port in the cornea for an injection or infusion of drugs.

The proximal end, or head of the shunt can be provided with a means, such as a color or shape for indicating shunt type. The distal end, or foot of the shunt can also be provided with a similar means, such as an indicator color, to more clearly show when the foot is properly positioned in the anterior chamber.

As noted above, the embodiment of the present invention can be provided as a transcorneal implant device to relieve intraocular pressure, or as a transscleral device to introduce medications into the posterior chamber of the eye. For example, as shown in FIGS. 30, 31 and 32, the implant device, or shunt 310 can be made from a hydrogel material which can absorb drugs, or it can be made from a porous material such as ceramic or titanium. It can also be a hydrogel material casing which encloses a porous material 312 containing a drug, wherein the hydrogel or porous material 312 releases the drug at a controlled rate (i.e. controlled diffusion) into the posterior chamber of the eye. The device 310 is anchored in the cornea or sclera by flanges 317 substantially as described above, and can also be anchored by a coating on the outside of the device. This coating can be porous or can be chemically modified to attract cellular attachment. The therapeutic agents or time-release drugs which can be released into the eye include any number of substances, including immune response modifiers, neuroprotectants, corticosteroids, angiostatic steroids, anti-glaucoma agents, anti-angiogentic compounds, antibiotics, radioactive agents, anti-bacterial agents, anti-viral agents, anti-cancer agents, anti-clogging agents and anti-inflammatory agents.

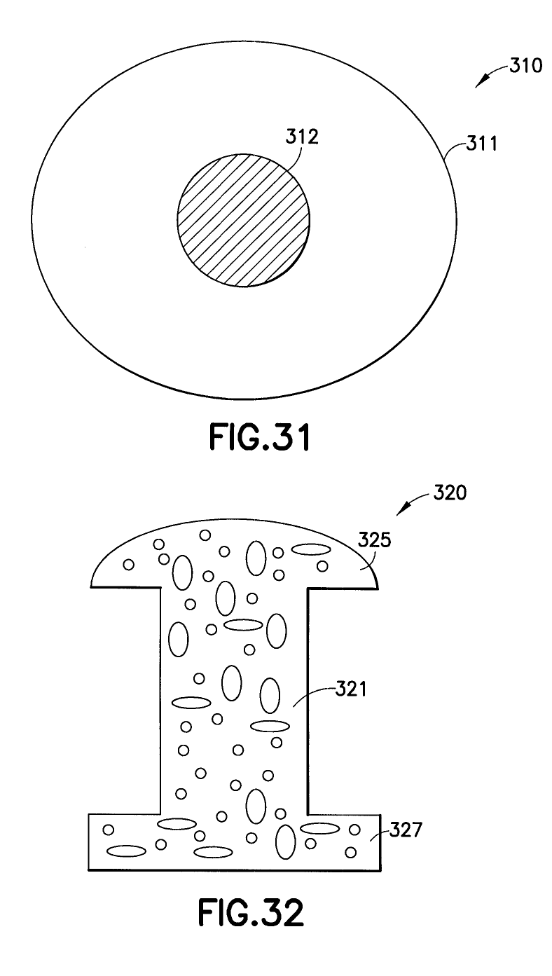

The embodiment of the invention shown in FIGS. 30 and 31 illustrates an example of a device having a hydrogel material casing which encloses a porous material 312, wherein the hydrogel or porous material releases the drug at a controlled rate into the posterior chamber of the eye. The device is implanted through the sclera and the drug is delivered slowly into the eye, and can be provided as a permanent or short term implant. As shown in FIG. 30, the implant can include a distal and proximal end, 317 and 315, respectively, between which a shunt body 311 extends. Fluid communication through the shunt is provided by an opening 314 extending between distal and proximal ends, and the opening can include a porous filter 312 containing a drug.

The outer surface of the shunt body 311 extending between distal and proximal ends can include an external layer or coating that is porous or chemically formulated to attract cellular attachment or growth. The outer surface of the shunt body 311 can also be provided with a porous layer or coating of titanium and/or ceramic wherein any required or additional drugs can be stored in the pores. The remainder of the shunt 310 can be constructed as a hydrogel casing.

The proximal end, or head of the shunt 310 can also be constructed of porous or non-porous hydrogel with a drug absorbed. In yet another embodiment of the present invention shown in FIG. 32, the entire shunt 320 can be constructed of a porous or nonporous hydrogel and can be provided without a filter.

The embodiment of the present invention described above is primarily provided as a long term implant which can be used to provide drug transmission to the eye over any number of prolonged periods. As such, the embodiment does not cause injury to the eye as does repeated injections, and yet allows a slow continuous infusion into the eye. Additional details of such a long term implant are noted in U.S. patent application entitled “Systems And Methods For Reducing Intraocular Pressure”, Ser. No. 10/182,833, and in U.S. Pat. No. 5,807,302, entitled “Treatment For Glaucoma”, the entire content of each being incorporated herein by reference.

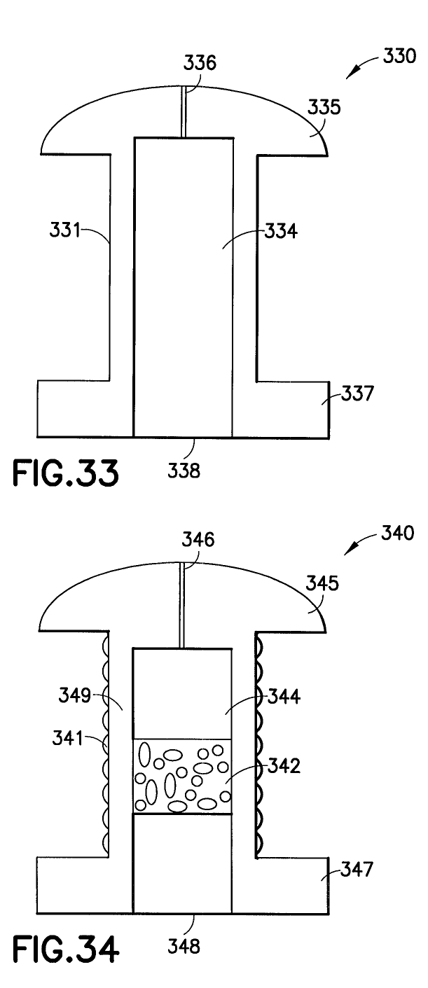

In yet another embodiment of the present invention shown in FIG. 33, the shunt 330 can be constructed as a porous flow control device which has an antibiotic or anti-infective agent. As described for each embodiment above, the device shunts aqueous humor from the anterior chamber to the tear film in order to reduce the intraocular pressure, or introduces a substance into the posterior chamber depending upon the application and shunt position. It can be placed through either the cornea or through the sclera with one end on the surface of the cornea, limbus or sclera, and the other end in the anterior or posterior chamber.

As shown in FIG. 34, the shunt 340 also includes a porous filter structure to provide a desired flow resistance required to drain the aqueous humor at a controlled rate. An anti-infective or antibiotic agent in the porous filter structure prevents bacteria infiltration from the outside of the eye through the filter 342 and into the anterior chamber. The exterior shunt body surface 341, which is in contact with tissue, can also have a porous or spongy texture to promote cellular ingrowth and help secure the device in the eye. The porous filtration device 342 provides an antibiotic or an anti-infective agent in a structure which prevents bacteria infiltration and decreases the risk of infection. The porous filtration device structure also provides a tortuous path to further prevent bacteria infiltration. The narrowed opening 346 located at the proximal end of the opening or channel 344 also provides a barrier to bacteria infiltration.

Existing applications typically incorporate a 0.20 micron pore size filter in a shunt for bacterial prevention. However, a 0.20 micron filter substantially restricts the flow through the device to such a great extent that the size of the filter area required to achieve the desired flow rate is not practical. If an antibiotic or an anti-infective agent is used in a structure with a larger pore size, the required flow resistance can be obtained in a much smaller device. Thus, where such an agent is used, the shunt can be smaller than any existing device which includes such a bacteria prevention mechanism. In addition, a porous structure with pore sizes greater than 0.2 microns will be less likely to become blocked than a device which uses a 0.2 micron filter as a means for preventing bacteria. A smaller device will also be less likely to cause irritation and rejection problems, and the device can be more easily positioned without disrupting the visual field or being overtly noticeable.

The porous nature of the device in areas where it is in contact with tissue also has the advantage of allowing cellular ingrowth, which aids tissue adhesion to the device and allows the device to be placed more securely in the eye. This helps prevent undesired extrusion after the device has been implanted.

As known to those skilled in the art, the flow rate in such devices is directly related to pore size. As noted above, existing filtration devices have had filters with pore sizes of approximately 0.2 microns in diameter to physically prevent bacteria from penetrating into the anterior chamber. A filter with this pore size restricts the flow excessively, thereby making the required filter area which is needed to achieve the required flow rate too large. This results in the working device being much larger than desired. If an antibiotic or anti-infective agent is added however, a filter with a larger pore size can be used having a similar or superior bacteria barrier response, and the desired flow resistance is obtained in a much smaller device.

Existing filtration devices that treat glaucoma by shunting fluid from the anterior chamber to the tear duct also have typically had no means of promoting cellular ingrowth to aid tissue adhesion to the device. The porous nature on the outside of the embodiments described above have the advantage of promoting cellular ingrowth which aids cell adhesion to the device and the device can be more securely held in place.

Some shunt concepts which drain aqueous humor from the anterior chamber to the tear film also include a valve mechanism, however, many have only a one way valve. Such a valve may not prevent all bacteria from infiltrating through the valve and thus the risk of infection is high. Therefore, the filtration devices of the embodiments described above solve this problem by also providing a tortuous path with an anti-infective agent through the filter 342 which kills bacteria before they can enter the anterior chamber.

The embodiments shown in FIGS. 34 through 36 include a porous metal, ceramic or plastic cylinder filter 342, 352 and 362, respectively, each with an outside diameter between approximately 0.010 and approximately 0.03 inches, and a length between approximately 0.020 and approximately 0.030 inches. The pore size is between approximately 0.20 and approximately 15 microns in diameter depending on the material, surface area and depth. The porous filter 342, 352 and 362 each have an anti-infective agent coated or compounded into its structure, which can be a silver compound, antibiotic or other broad-spectrum anti-infective agent, which is biocompatible. The filter depth also provides a tortuous path with the agent coating or compound which can prevent bacteria from infiltrating for an extended period.

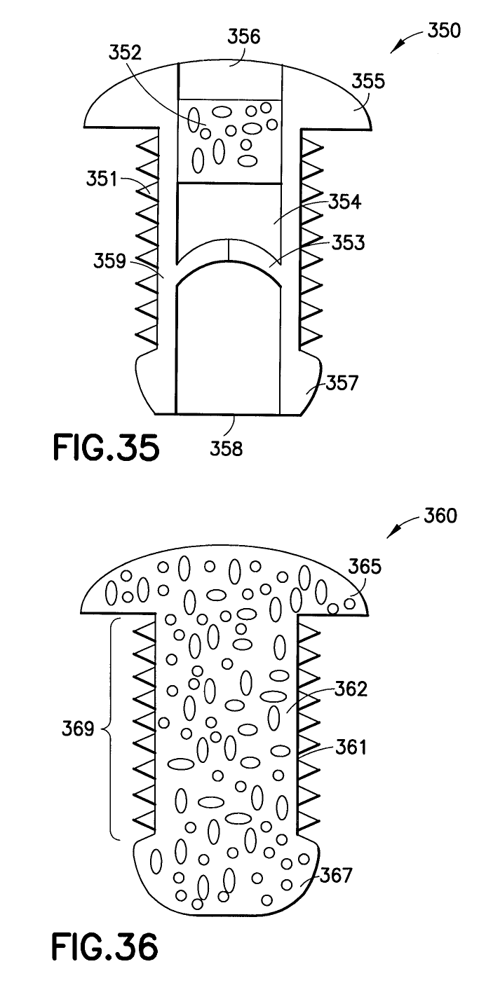

In FIGS. 34 and 35, the cylindrical filter 342 and 352, respectively, is enclosed in a silicone or hydrogel tube or channel 344 and 354, respectively, which at a proximal end 345 and 355, respectively, has a smooth curved flange which conforms to the surface of the eye like a contact lens, but which has an opening 346 and 356, respectively, through which aqueous humor can flow. The distal end 347 and 357, respectively, has a flange which secures the device and prevents extrusion. The outside tube 341 and 351, respectively, protects the tissue from toxic effects of the anti-infective agent and is made from a soft material. As with the embodiments described above, the part of the tube that contacts tissue can have a spongy texture so that cellular ingrowth can occur. Also, as shown in FIG. 35, a valve 353 can be provided to control the flow rate through the porous filter structure 352 which further incorporates the anti-infective agent. Still other embodiments of valves can include ‘poppit-type’ valves, ‘blow-off’ type valves, user activated valves, Vernay™-type valves, duck-bill valves, umbrella valves, pressure cracking valves and dome-over valves, as known to those skilled in the art.

Also as described above, a totally porous ceramic part 360 can be constructed with an impregnated biocide as shown in FIG. 36. The ceramic is a bioinert, bioactive, and/or biocompatible material such as alumina or hydroxyapitite. The anti-infective agent used is also bioinert in the quantities needed, such as a silver compound or an increased concentration of the eyes natural anti-infective agents.

The shape of the shunt 360 can be similar to those described above, and may also include a series of mechanical engagement threads 369 as shown in FIG. 36 to hold it in the tissue like a mechanical screw. Yet another engagement technique can use a number of protrusions, such as detents, indentations or tabs (not shown) for fixation in the tissue.

The totally porous, ceramic part can be constructed with pore sizes of approximately 0.2 microns. In this embodiment, the device can control the flow resistance, provide the outside biocompatible structure, and prevent bacteria infiltration due to pore size in a single, integral device, without requiring a valve channel and/or separate filter structures. The structure of the ceramic part can also be made with an even larger pore size for greater flow rates, and a very thin layer sprayed or deposited onto the surface (e.g., approximately 0.2 micron). A totally porous titanium part can also be constructed into the above shapes using a sintering process with an impregnated biocide.

In the embodiments described above, the shunt, implant, or filter therein, is constructed based upon a relationship between pore size and the flow rate. The larger the pore size the greater the flow rate in a device. This enables a very small device to be made which can effectively control the flow of the glaucoma filtration device. Added benefits include the use of an anti-infective agent to kill bacteria and prevent their infiltration. The anti-infective agent can be used in cooperation with the tortuous path structure created by the porous materials. Also, the use of a porous structure further enables cell ingrowth and promotes cell adhesion to the surface of the device when implanted in the human body.

The above device can also be used as a drug delivery device. Specifically, the above embodiments can include drugs in the porous filter or body materials which dissolve over time and are released into the eye. In still another application, the device can be used as a mechanism to inject drugs into the eye (i.e., a catheter). This can be a temporary implant or an ophthalmic catheter. Related material is disclosed in U.S. Pat. No. 5,807,302, entitled “Treatment of Glaucoma”, in U.S. Pat. No. 3,788,327, entitled “Surgical Implant Device”, in U.S. Pat. No. 4,886,488, entitled “Glaucoma Drainage the Lacrimal System and Method”, in U.S. Pat. No. 5,743,868, entitled “Corneal pressure-Regulating Implant Device” and in U.S. Pat. No. 6,007,510, entitled “Implantable Devices and Methods for Controlling the Flow of Fluids Within the Body”, the entire content of each being incorporated herein by reference.

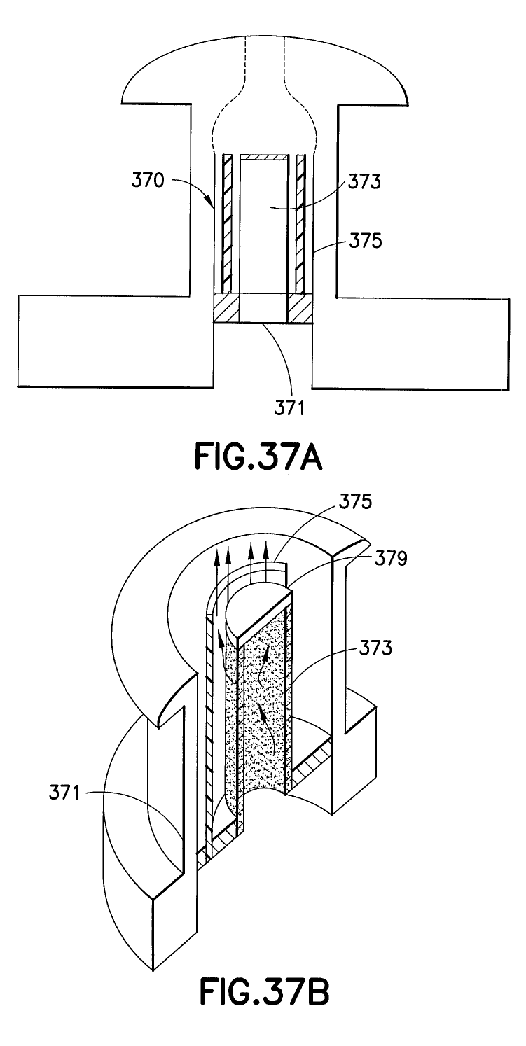

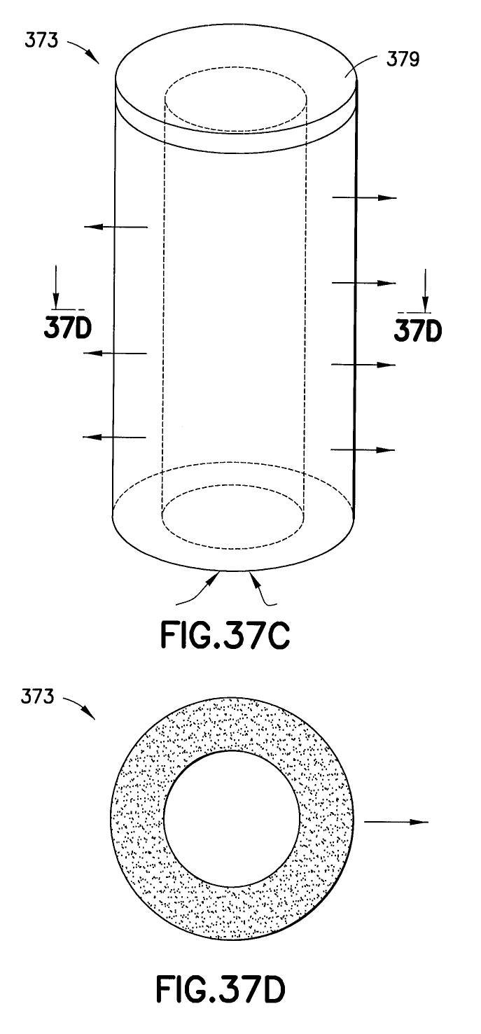

In yet another embodiment of the porous bodies or filters in the above devices, a hollow or capillary action micro-device can be provided as shown in FIGS. 37 through 42. The fluidic micro-devices of FIGS. 37 through 42 are designed to be part of the pressure release insertion device, implants or shunts described above, and can serve as a check valve to release elevated pressures in the eye.

As shown in FIGS. 37A through 37D, the hollow or capillary action micro-device 370 can consist of an elongated porous filter, constructed having a potted base 371 which secures at least one hollow, porous fiber 373 surrounded by a plastic cylinder 375 within the channel of the implant or shunt. The fiber can be closed or sealed at a first end 379 and is open and secured to a fluid communication opening within the base 371 at a second end. Throughout the length of the fiber 373, a porous wall surrounds a substantially hollow center, and extends within the plastic cylinder along the axis of the shunt. The porous fiber creates a much larger filtering area for the micro-device 370, and unrestricted flow is then provided via the surrounding plastic cylinder 375, hollow fiber center and the communication opening within the base 371. The fiber construction therefore, provides a maximum flow via the restrictive porous openings along the length of the fiber.

The use of hollow, porous fiber technology can be used to increase the effective filtering area provided when inserted into the implant bodies described above. Aqueous travels into the shunt channel and through the open end of the base 371 and into the substantially hollow center of the fiber 373. As the fiber is closed at the opposite end 379, the aqueous is forced to pass through the porous layers of the fiber to escape the fiber 373. The aqueous then enters the plastic cylinder 375 and thereafter exits the shunt channel to the surface of the eye. As shown in greater detail in FIGS. 37C and 37D, the hollow fiber filter 373 provides a substantially cylindrical element, closed at a first end 379. As aqueous enters the substantially hollow center via the opposite open end of the fiber 373, it must exit through the porous materials of the fiber body. These pores of the fiber 373 can be uniform over the fiber body, or can be provided having a gradient pore size, from small to large as measured radially out from the center of the fiber.

The potted base 371 can be comprised of a substantially circular disk having a diameter of approximately 0.020 inches, and includes at least one opening in communication with the hollow, porous fiber 373 secured to and extending from the opposite side of the base as shown in FIGS. 37A and 37B. A length, inside diameter and porous wall configuration (i.e., pore size and gradient) of the fiber 373 can be configured to achieve the desired filter/restriction result required by the application. Additionally, construction materials can include materials as those described above to assist in achieving the desired results. As described in greater detail below, the hollow or capillary action micro-device can also be implemented as a bonded two piece member to achieve substantially the same results.

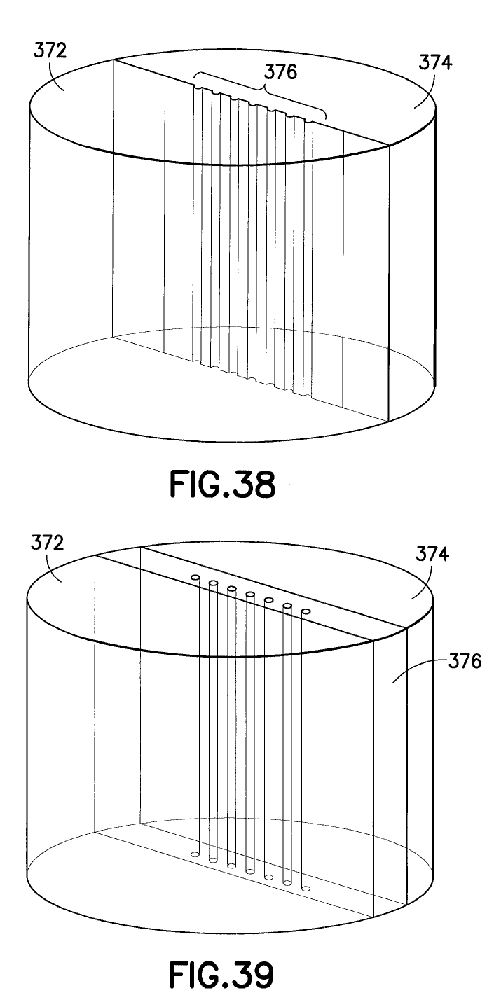

As shown in FIGS. 38 and 39, another hollow or capillary action micro-device can consist of two or more separate parts 372 and 374, which are bonded together. As known to those skilled in the art, the bonding can be done using laser welding techniques with wavelengths in the range from approximately 800 nm to over 1,000 nm. In at least one part of the device, a maze of capillary vessels 376 are implanted or imbedded. The capillary vessel dimensions and their geometry are calculated and manufactured to satisfy required parameters for relieving pressure in the eye.

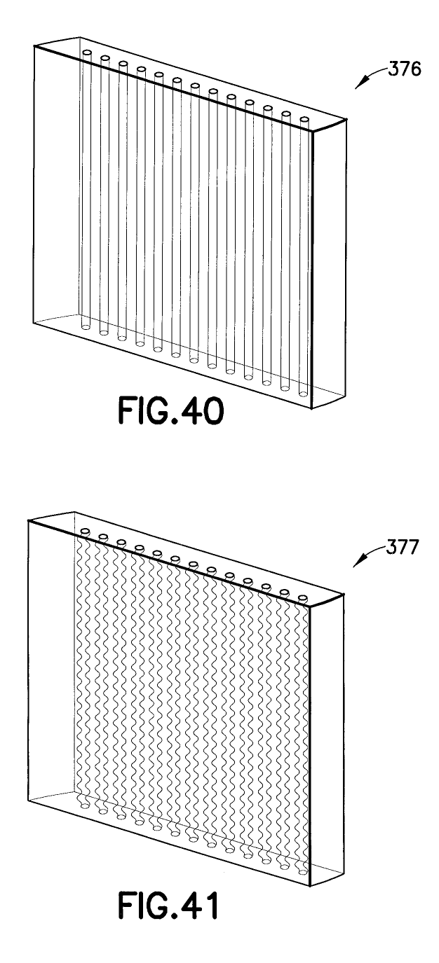

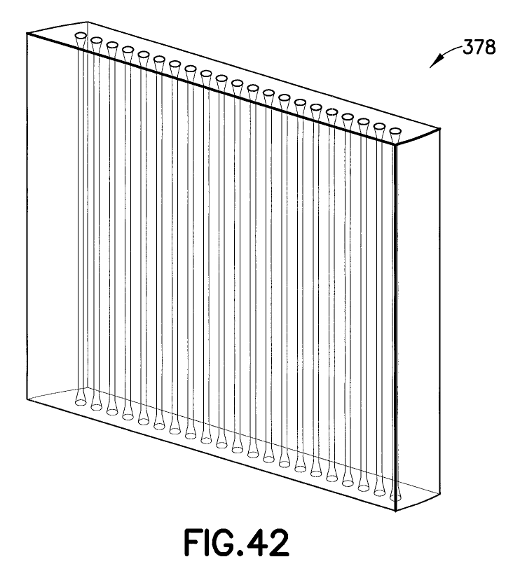

As shown in FIG. 40, the capillary vessels of member 376 can be constructed having a straight profile extending the entire length of the member, and are formed having a diameter of approximately 0.001 mm. In FIG. 41, another variation of the capillary member is shown, wherein the capillary vessels of member 377 are shown having a substantially sinusoidal wave shape extending the entire length of the member, and are formed having a diameter of approximately 0.001 mm. In FIGS. 40 and 41, the capillary members can be further constructed having an expanded portion along a longitudinal axis (not shown), wherein a substantial portion of the capillary members can be used to provide a reservoir. In another variation of the capillary member shown in FIG. 42, the capillary vessels of member 378 have a straight profile where extending through the reservoir section. However, near opposite ends, the capillary vessels can be reduced in diameter, or constructed having an enlarged conical orifice at one or both ends, thereby controlling resistance at the device.

Each part of the device 372, 374, 376 and 378 can be molded using a master provided by a technique such as photolithography, allowing construction of capillary members with accurate sub-micron dimensions. Such devices provide a very high level of repeatability and reliability.

Still other embodiments can include a capillary member having a wick member (not shown) positioned within the capillary orifice. In such an embodiment, a capillary action wick can be constructed using any number of materials, such as carbon, glass, polypropylene fiber, metallic silver or crimped fiber bundles.

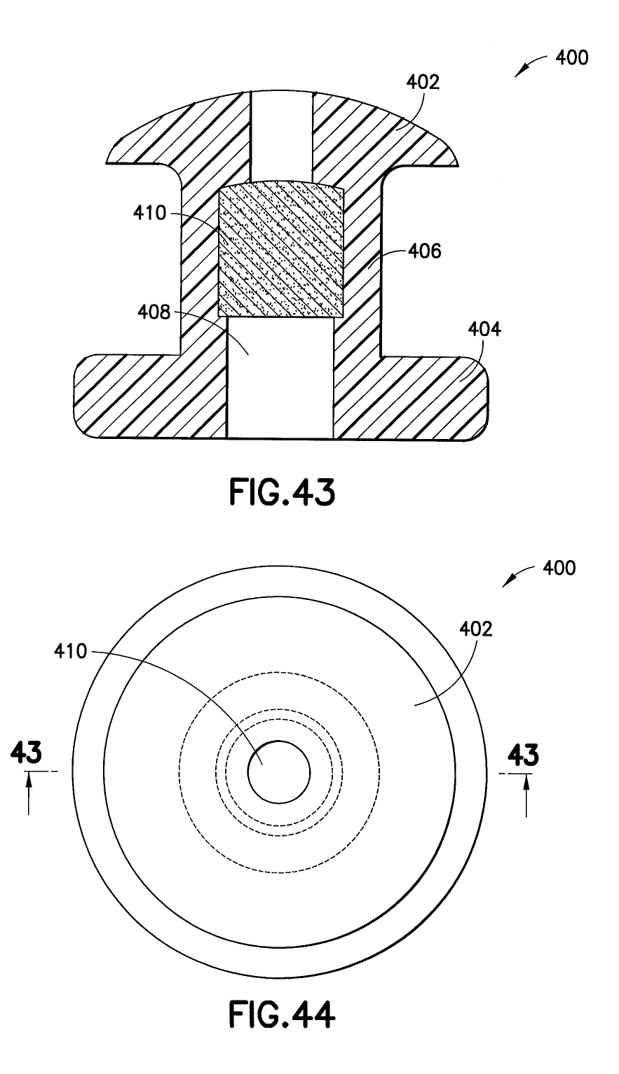

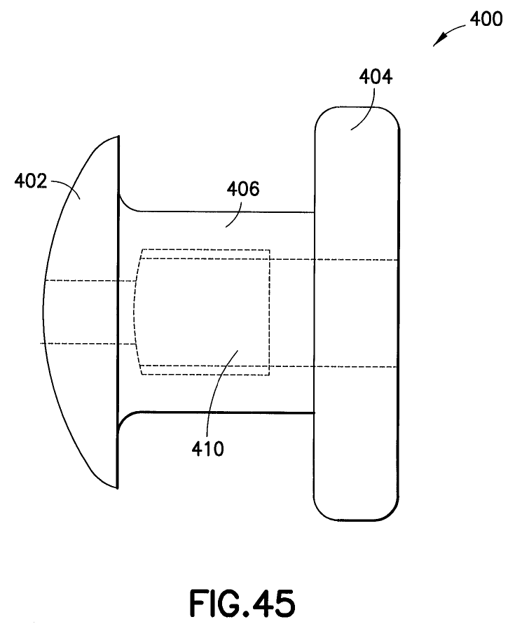

FIGS. 43 through 45 illustrate another embodiment of the present invention in which each above feature or features can be provided. The shunt 400 shown provides a head 402, foot 404 and body 406 therebetween having a channel 408 for fluid communication between opposite ends. The device can be constructed using any of the construction materials outlined above, and includes a filter and/or valve assembly 410 incorporating any of the improved techniques specified above.

The preferred embodiment of the shunt 400 consists of a polymeric hydrogel housing 406 and can include a sintered titanium flow-restricting filter 410. The shunt housing 406 is approximately 1.5 mm long and has a cylindrical central section with flanges 402 and 404 at each end. The proximal, or external flange or head 402 is approximately 1.4 mm in diameter and has a semispherical profile to make it less detectable to the eyelid. The distal, or internal flange or foot 404 anchors the shunt 400 within the cornea. As described in greater detail below, in a first and second variation of the embodiment shown, two different central section lengths (e.g., 0.76 mm and 0.91 mm in the dehydrated state) can be provided to accommodate various corneal thickness.

The shunt housing 406 can be made of ocular hydrogel (i.e., poly hydroxyethyl methacrylate-methacrylic acid copolymer (polyHEMA-MAA) polyHEMA, copolymers and other expansion material hydrogels), having distinct hydrated and dehydrated states. For example, water content in a hydrated state can be approximately 40 to 45%. The primary material, polyHEMA, is commonly used in vision correction devices such as soft contact lenses, and is rigid in the dehydrated state. When hydrated, the material swells by approximately 20% (i.e., specifically, between approximately 10% and approximately 50%), and becomes soft and pliable. These properties, as provided by the manufacturing steps described below, allow the shunt 400 to be implanted in the dehydrated state to take advantage of its rigidity, and transition to a hydrated state once in position allowing it to become soft and compliant after implantation.

The shunt 400 can be manufactured by casting a monomer mixture comprising HEMA, methacrylic acid and dimethacrylate crosslinker into a silicone mold and heat-curing the mixture to create a hydrogel rod. The rod is then de-molded and conditioned under elevated temperature. The rod is finally machined into the shunt casing geometries defined in greater detail below.

The filter/restrictor member shown in use with the example embodiment, is a sintered titanium flow restrictor 410 which allows controlled passage of aqueous humor from the anterior chamber to the tear film. Titanium has a long history of safety in implantable devices such as orthopedic devices, pacemakers, arterial stents and artificial hearts. The flow restrictor example 410 is manufactured by pressing finely graded titanium powder in a mold and applying heat to sinter the individual particles together, resulting in a porous structure with thousands of random labyrinthine fluid pathways that limit the flow rate to a level appropriate for effective IOP reduction. Such a process can include metal injection molding, in which a binder is included with a round material, such as titanium powder or ceramic, to create a series or graduation, of pore sizes.

A second function of the flow restrictor 410 is to aid in preventing bacterial ingress. The same labyrinthine fluid pathways that limit the outflow of aqueous humor from the eye are also intended to serve as a barrier to inhibit bacteria ingress. For the titanium flow restrictor shown used in this embodiment, a flow rate between approximately 1 to 6 ul/min at 10 mm Hg is provided. Still other flow rates can be provided using the restrictor/valve configurations described above.

The shunt 400 is typically implanted into an approximately 1.6 mm incision in the cornea while in a dehydrated state. The 1.6 mm incision is created approximately 1 to 2 mm from the superior limbus. The shunt flange to flange lengths are designed to be implanted at that location, and this ensures that the shunt 400 is covered by the upper eyelid and does not affect the patient's field of vision. Cornea thickness variations between patients is taken into account by providing different size shunts. Specifically, the shunt is available in two or more different central section lengths (e.g., flange-to-flange length), between approximately 0.5 mm and approximately 1.0 mm (e.g., 0.76 mm and 0.91 mm in the dehydrated state) to accommodate various corneal thickness at the location of 1 to 2 mm from the superior limbus. This ensures that there is a good fit in the cornea and the extra length in the shunt in a thin cornea does not hit the iris.

The foot 404 size is provided so that extrusion of the device while implanted is minimized. The foot size enables the shunt to be implanted into the incision in its dehydrated state and then seal the incision after hydration while also minimizing extrusion of the device long term. The foot 404 diameter is approximately 0.031 inches greater in diameter than the central shaft of the housing 406 in its hydrated state to achieve this goal. The hydrated and dehydrated dimensions, in relation to one another and an incision size as described in greater detail below, are carefully prepared to create a number of optimized dimension ratios for the shunt to prevent extrusion, prevent leakage and prevent intrusion.

When in a dehydrated state, the head 402 is approximately 0.047 inches in diameter, the foot 404 is approximately 0.057 inches in diameter and the body extending between each is approximately 0.029 inches in diameter. After implantation the shunt 400 swells by approximately 20% to the hydrated dimensions and this hydration seals the 1.6 mm incision. Shunt foot 404 dimensions change from approximately 0.057 inches in its dehydrated state, to 0.065 inches in its hydrated state to prevent extrusion and leakage. The head 404 increases to approximately 0.055 inches to prevent intrusion, and the body extending between each expands to approximately 0.034 inches in diameter to further prevent leakage.

In the current application example, in which a 1.6 mm incision is prepared, the preferred embodiment of the shunt includes a foot diameter/body diameter ratio (i.e., an optimized dimension ratio), in a hydrated state of between approximately 1.3 and approximately 3.0, with a desired value of approximately 1.91. To establish this value in this shunt embodiment, the foot 404 is constructed to have a diameter approximately 0.016 inches larger than the body diameter in the hydrated state.

As noted above, in this application example a 1.6 mm (0.063 inch) incision is prepared. Therefore, another optimized dimension ratio can be established between the incision size and the foot size in the hydrated and dehydrated states. The preferred embodiment of the shunt includes an incision size/foot diameter ratio (i.e., an optimized dimension ratio), in a dehydrated state of between approximately 1.0 and approximately 1.3, with a desired value of 0.063/0.057=1.10.

The preferred embodiment of the shunt can also include an incision size/foot diameter ratio in a hydrated state (i.e., after implantation) of between approximately 0.75 and approximately 1.0, with a desired value of 0.063/0.065=0.97. In doing so, the foot diameter is larger than the incision length after hydration to prevent extrusion and leakage.

The preferred embodiment of the shunt can still further include an incision size/body diameter ratio in a hydrated state (i.e., after implantation) of between approximately 1.25 and approximately 2.0, with a desired value of 0.063/0.034=1.85. In doing so, the body diameter increase after hydration helps prevent leakage. Still another benefit of an increased body diameter is the elimination of any sutures required to close the incision or secure the shunt, making the procedure much quicker.

The change in material properties from a hard rigid device in its dehydrated state to a soft pliable device in its hydrated state provides a number of advantages. When the device is hard and rigid in its dehydrated state, the implantation procedure is easier and there is less chance of damaging the shunt or dislodging the filter. When the shunt hydrates, the material becomes soft and pliable. The soft and pliable nature of the device upon hydration ensures comfort for the patient and it minimizes stress to the cornea and eyelid, which are very sensitive.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

1. An ocular implant for fluid communication with an anterior or posterior chamber of an eye, comprising:
a body having a distal end and a proximal end, the body extending from at least one of an anterior and a posterior chamber to an outer surface of an eye;
a head defining an opening and positioned at the proximal end of the body for engagement against the outer surface of the eye;
a foot defining an opening and positioned at the distal end of the body for engagement within at least one of an anterior and posterior chamber;
a channel in the body communicating with respective openings in the head and the foot; and
a flow restrictor in the channel located at an intermediate position, disposed distally with respect to the head and proximally with respect to the foot; and
a flap, in a normally closed position, that opens in response to a minimal pressure, wherein the flap is disposed proximally with respect to the flow restrictor;
wherein the implant is inserted in a dehydrated state in which the implant in a substantially rigid form and the implant is hydrated after insertion, providing the implant in a substantially soft and pliable form.

2. The implant of claim 1 wherein the flap is a one-way valve.

3. The implant of claim 1 wherein the flap is positioned within the head, contiguous with the opening in the head, wherein the opening of the head has a smaller cross section than the channel.

4. The implant of claim 1 further comprising a selected marking based, at least in part, on the size of the implant.

5. The implant of claim 1 further comprising a drug, wherein the body encloses the drug for controlled release.

6. The implant of claim 1 wherein the body has a star-shaped cross section.

7. The implant of claim 1 further comprising a micro-mechanical pump mounted in the body.

8. The implant of claim 1 further comprising a biocide on the flow restrictor.

9. The implant of claim 1 wherein at least one of the body, the head and the foot is constructed of at least one of an ocular hydrogel having a dehydrated state and a hydrated state, silicone, polymethylmethacrylate (PMMA), poly 2-hydroxyethylmethacrylate (PHEMA), hylauronic acid, a silicone/hydrogel combination, a silicone acrylic combination, fluorosilicone acrylate, ceramic, coral and stainless steel.

10. The implant of claim 9 wherein the hydrated state provides at least one of a body, head and foot dimension that is approximately 10 to approximately 50 percent larger than the dehydrated state.

11. The implant of claim 1 wherein the foot comprises a circular cross section and the body comprises a circular cross section, and
wherein a ratio between a diameter of the foot circular cross section and a diameter of the body circular cross section is defined as foot circular cross section diameter divided by body circular cross section diameter, and
wherein the ratio between a diameter of the foot circular cross section and a diameter of the body circular cross section has a value of between approximately 1.3 and approximately 3.00.

12. The implant of claim 1 wherein the flow restrictor includes a porous fiber adapted to permit fluid flow therethrough, and a cylinder surrounding the porous fiber.

13. The implant of claim 1 wherein the flow restrictor is a capillary flow restrictor.

14. The implant of claim 13 wherein the capillary flow restrictor defines capillary vessels having a sinusoidal wave shape.

15. The implant of claim 1 wherein the foot is a linear distal member.

16. An ocular shunt comprising:
a body having a distal end and a proximal end;
a head comprising a flange positioned at the proximal end of the body and defining an head opening;
a foot comprising a flange disposed at the distal end of the body and defining a foot opening;
a channel in the body communicating with head opening and the foot opening; and
a flow restrictor in the channel located at an intermediate position, disposed fully distally with respect to the head and fully proximally with respect to the foot;
wherein the implant is constructed of an ocular hydrogel having a dehydrated state and a hydrated state, and wherein the hydrated state provides at least one of a body, head and foot dimension that is approximately 10 to approximately 50 percent larger than the dehydrated state, and
wherein the flow restrictor is made of one of the group consisting of metal, titanium, ceramic and glass.

17. The shunt of claim 16 further comprising a structure adapted to control the flow of fluid through the channel, in fluid communication with the channel and responsive, at least in part, to pressure in the proximal direction to thereby permit fluid flow in the proximal direction in response to the pressure.

18. An ocular shunt comprising:
a body having a distal end and a proximal end;
a head comprising a flange positioned at the proximal end of the body and defining an head opening;
a foot comprising a flange disposed at the distal end of the body and defining a foot opening;
a channel in the body communicating with head opening and the foot opening; and
a plurality of the filters arranged in a predetermined order disposed in the channel and located at an intermediate position, disposed distally with respect to the head and proximally with respect to the foot;
wherein the implant is constructed of an ocular hydrogel having a dehydrated state and a hydrated state, and wherein the hydrated state provides at least one of a body, head and foot dimension that is approximately 10 to approximately 50 percent larger than the dehydrated state; and
a coating applied to at least one of the head, the foot, the body and the filter.

19. The shunt of claim 18 wherein the coating is a surgical adhesive applied to at least one of the head, the foot, the body.

20. The shunt of claim 18 further comprising a flap in fluid communication with the channel and selectively openable to permit fluid flow through the head opening.
